(19) United States
(12) Patent Application Publication
(10) Pub. No.: US 2025/0155401 A1
(43) Pub. Date: May 15, 2025

(54) RAPID, SINGLE-STEP AND REAGENTLESS ELECTROCHEMICAL BIOSENSING METHODS, COMPOSITIONS AND DEVICES FOR NANOMOLAR DETECTION OF C-REACTIVE PROTEIN

(71) Applicant: Advanced Biomedical Instrumentation Centre Limited, Hong Kong (HK)

(72) Inventors: William Lawrence WHITEHOUSE, Hong Kong (HK); Hoi Ying Louisa LO, Hong Kong (HK); Julian Alexander TANNER, Hong Kong (HK)

(21) Appl. No.: 18/916,683

(22) Filed: Oct. 15, 2024

Related U.S. Application Data
(60) Provisional application No. 63/597,706, filed on Nov. 10, 2023.

(51) Int. Cl. G01N 27/327; G01N 33/543

(52) U.S. Cl. CPC G01N 27/3277; G01N 27/3276; G01N 33/5438

(57) ABSTRACT

An electrochemical biosensing method using screen-printed gold electrodes functionalized with a redox probe modified DNA aptamer that binds specifically to CRP is provided. Binding-induced conformational switching of the CRP-targeting aptamer induces a specific and selective signal-ON event, which enables single-step and reagentless detection of CRP in as little as 1 minute. The aptasensor limit of detection spans approximately 20-60 nM in 50% human serum with dynamic response windows spanning 1-200 or 1-500 nM (R=0.97/R=0.98 respectively). The sensor also operates in undiluted human blood producing an LOD of 45 nM equating to 5.4 mg/L. The sensor is stable for at least 1 week and can be reused numerous times, as judged from repeated real-time dosing and dose-response assays.

### CROSS-REFERENCE TO RELATED APPLICATIONS

The present application claims priority from U.S. provisional patent application Ser. No. 63/597,706 filed Nov. 10, 2023, and the disclosure of which is incorporated herein by reference in its entirety.

### REFERENCE TO SEQUENCE DISCLOSURE

The sequence listing file under the file name “P2987US01_SEQ LISTING.xml” submitted in ST.26 XML file format with a file size of 4 KB created on Oct. 15, 2024 and filed on Oct. 15, 2024 is incorporated herein by reference.

### FIELD OF THE INVENTION

The present invention generally relates to the field of biotechnologies. More specifically the present invention relates to a rapid electrochemical biosensing system and method for detecting C-reactive protein.

### BACKGROUND OF THE INVENTION

The development of rapid and easy-to-use point-of-care devices are sorely needed to improve prognosis in the clinic, especially for pathological states such as sepsis where acute response to infection escalates quickly. Large cohort studies have reported prognosis severity for sepsis to increase as much as 10% per hour between patient admission and administration of antibiotics.

C-Reactive Protein (CRP) is used as a well-established biomarker for clinical assessment of infection and acute-phase immune response to infection. CRP measurements from blood or serum usually require laboratory-based instrumentation such as plate readers, incubators and/or light sources, light-scattering optics and photometers. Immunoassay-based methods such as ELISA have improved considerably over the last decade. However, analytical techniques used in this context are still limited by the time required to produce results, typically 2-3 hours for ELISA, without considering backlogs that are common in centralized clinical laboratories. Such methods must also be conducted by laboratory-based specialists using large equipment and typically require multiple reagents and/or labels to generate signal readouts for analyte quantification, all of which add to operating costs.

To overcome these limitations, much effort has been spent on development of electrochemical sensors that can be more easily miniaturized and operated by non-specialists hence proving great promise for translation into point-of-care (POC) technologies. The glucose monitor is the only commercially available bioelectronic sensor to date yet proves the immense value of such devices and paves the way for future innovations especially for application in the clinic to reduce sample submission to result times significantly. In the context of sepsis, rapid results can improve prognosis significantly hence promoting the development of electrochemical POC devices to achieve these aims. In this context, aptamers used as recognition components for electrochemical biosensing are gaining significant interest and compromise a unique class of sensors, the so-called E-DNA sensors (electrochemical-DNA sensors). This term is interchangeably used alongside electrochemical-aptamer-biosensors “EABs” or “Aptasensors” more generally. Compared to antibodies, aptamers can be synthetically manufactured on mass-scales with greater reproducibility and significantly reduced costs. Crucially, aptamers can maintain and even rival sensitivities of antibody-based detection of protein markers. Aptamers can also target a wider range of analytes, possess improved chemical and thermal stability and can be more easily immobilized onto a wide range of surfaces used in biosensor development, or conjugated for detection without loss of activity often observed when labelling or immobilizing antibodies.

CRP fluctuation is also indicated across a wide range of chronic conditions including but not limited to Osteomyelitis, inflammatory bowel disease (IBD) and some forms of arthritis. Measurement of CRP can aid in the assessment or progress of such conditions outside of the clinic, yet no commercially viable sensor is available for use by the general-public. It is believed that such a device has the potential of being significantly beneficial to public health in the same way as the highly successful glucose sensors are, and the present invention addresses this need.

### SUMMARY OF THE INVENTION

It is an objective of the present invention to provide a device, composition, or method to solve the aforementioned technical problems.

In accordance with a first aspect of the present invention, a rapid, single-step and reagentless electrochemical biosensor system for detecting C-reactive protein (CRP) is provided. Particularly, the system includes a screen-printed gold working electrode with redox-tagged CRP-targeting aptamer immobilized on the surface of the gold working electrode and back-filled with alkane-thiols to prevent non-specific adsorption, a platinum counter-electrode, a ceramic substrate, a current signal recorder connected to the screen-printed gold working electrode and the platinum counter-electrode, and a data processor for receiving the current signals from the current signal recorder and processing the current signals.

Specifically, the dynamic range of the electrochemical biosensor system is 1 mg/L to 50 mg/L in undiluted human blood.

In accordance with one embodiment of the present invention, the CRP detection sensitivity of the system is up to detecting a CRP concentration of 5.4 mg/L in human blood.

In accordance with another embodiment of the present invention, the redox tag of the CRP-targeting aptamer comprises methylene blue.

In accordance with other embodiment of the present invention, the redox-tagged CRP-targeting aptamer is immobilized on the surface of the gold working electrode by thiolation of the aptamer and forming a gold-thiol bond between the thiolated aptamer and the surface of the gold working electrode.

The CRP detection of the electrochemical biosensor system is, in particular, no longer than 10 minutes, and is reusable for more than 1 time for dose-response assays.

In accordance with a second aspect of the present invention, a rapid, single-step and reagentless electrochemical biosensing method for detecting CRP in a specimen is provided. Specifically, the method includes steps of collecting a specimen from a subject; obtaining a serum from the specimen, subjecting the specimen to the electrochemical biosensor system as described above; and determining the presence or absence of CRP in the specimen by observing the current signals received by the data processor.

In accordance with one embodiment of the present invention, the serum is CRP-positive if the current signal is increasing or decreasing in a dose-dependent manner.

In accordance with another embodiment of the present invention, the detection time for the CRP is no longer than 10 minutes.

### BRIEF DESCRIPTION OF THE DRAWINGS

Embodiments of the invention are described in more details hereinafter with reference to the drawings, in which:

FIG. 1 depicts the sensor fabrication and working principle, in which the left part schematically illustrates the aptamer electrochemical biosensor working principle that the DNA aptamer sequence is induced to switch conformation which brings the electroactive redox methylene blue component closer to the electrode surface upon binding to target CRP, the center part depicts that signal-ON response is measured from the current output using Square Wave Voltammetry (SQWV), and the right part depicts the predicted secondary structure of the CRP-targeting aptamer used in this study is shown with ‘ss’ count scoring where red color denotes the bases most likely to be single-stranded in presumed loop configurations.

FIGS. 2A-2D depict the sensor fabrication process, in which FIG. 2A shows the schematic illustration of aptamer with dual chemical modifications that the 5′disulfide group is chemically reduced with TCEP to produce a thiol moiety used to immobilize aptamers via gold-thiol bond formation, whilst a methylene blue redox molecule is tethered at the aptamer 3′ end to enable electrochemical signal induction, FIG. 2B shows the aptamer concentration of CRP-targeting aptamer and a non-thiolated control before and after immobilization, assessed via UV 260 nm absorbance from n=3 sensors, and FIGS. 2C and 2D display the corresponding Nyquist plots generated by EIS showing charge transfer before and after backfilling with MCH. In comparison to thiol-terminated aptamer, EIS interrogation reveals significantly reduced Rct for the control sequence after back-filling with MCH.

FIGS. 3A-3E shows the sensitivity and selectivity of CRP biosensing method, in which FIG. 3A depicts the optimization of applied frequency for Square-Wave-Voltammetry based electrochemical interrogation, along with specificity testing against a control aptamer sequence, FIGS. 3B-3C depict the current-voltage (IV) plots from n=4 sensors, prepared with CRP-targeting aptamer (FIG. 3B) and a sequence analogue where flanking primer regions are removed (FIG. 3C), FIG. 3D shows the dose response assay conducted in BSA doped PBS(1×) buffer confirmed specificity by comparison to the sequence control, and FIG. 3E demonstrates the dynamic sensing range used for linear regression is highlighted. Particularly, calibration plot using the dynamic range identified from dose-response assay is used for linear regression analysis.

FIGS. 4A-4D depict the sensitivity and recovery assessment in BSA doped PBS assay buffer, in which FIG. 4A shows the current-voltage plot of data used to generate response curves and calibration plots, FIG. 4B depicts the dose-response plots for initial sensor use (blue) and re-use (black) after 48-hours, FIG. 4C exhibits the calibration plot used for linear regression analysis, and FIG. 4D shows the log-10 scale plot used for non-linear regression analysis with fitting to Hill-Langmuir (dose-response) binding model.

FIGS. 5A-5D depict the sensor optimization and selectivity verification in 20% diluted human serum, in which FIG. 5A shows the current-voltage ‘IV’ plot showing sensor baseline drift in 20% human serum, particularly, a significant drift is observed for the sensor prepared with CRP-targeting aptamer in comparison to control sensors prepared using a shorter IL-6 specific aptamer, FIG. 5B depicts the corresponding peak height plot as function of time from n=4 electrodes, FIG. 5C shows the sensor stabilization after initial equilibration in 20% human serum and an additional blocking with BSA significantly minimizes baseline drift, and FIG. 5D shows the optimization of applied frequency for SQWV measurements, where the signal gain is significantly enhanced at higher above 500 Hz.

FIGS. 6A-6E depict CRP biosensing sensitivity maintained in human serum. FIGS. 6A, 6B and 6E are plots of relative response against the concentration and logarithmic concentration of C-reactive protein respectively, and the results of the experimental repeats, which show that the sensitivity is maintained in the nanomolar range, determined by both linear and nonlinear regression across experimental repeats. FIGS. 6C and 6D display that the additional proteins, including the sepsis specific marker procalcitonin (PCT) are spiked into human serum to verify the response nature to CRP.

FIG. 7 shows the real-time CRP detection in human serum through spiking recombinant (human cell expressed) CRP is spiked into droplets of human serum on screen printed electrodes, repeated electrode surface washing over 10 cycles with 100 nM CRP and a final spike of 10 nM CRP. The plots on the lower row correspond to the 4^{th} and 5^{th} cycles of washing with 100 nM CRP (left and center) and the final 10 nM CRP spike (right) respectively.

FIG. 8 shows the evaluation of re-usability and additional selectivity of the CRP biosensing system, over repeated uses for detection of different concentrations of CRP without displaying observable deviations in response.

FIG. 9 is a schematic diagram showing the electrochemical biosensor system of the present invention, comprising the biosensor 100 consisting of a platinum working electrode 101 and screen-printed gold working electrode 102 surfaced with immobilized CRP-targeting aptamers fixated on a ceramic substrate 103. The electrodes 101 and 102 are connected to a current signal recorder 200, which records the current readings and relays to the data processor 300 for signal visualization and further data processing.

FIGS. 10A-10C show the sensor response interrogation in human blood samples. FIG. 10A shows the current voltage plots averaged from N=6 sensors showing optimal applied frequency for sensor signal-ON and signal-OFF response. Confidence of variation values are derived and shown for each state. FIG. 10B shows the dose response assay; and FIG. 10C shows the corresponding calibration plot.

### DETAILED DESCRIPTION

In the following description, devices, systems, and/or methods of detecting CRP in electrochemical biosensing manner and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Recently, numerous efforts have been made to advance EABs by decoupling the response induction mechanism from simple adsorption-based methods (to induce signal output). By combining DNA nanotechnology advances with signal amplification techniques, a generalized aptamer-based biosensing platform has been successfully developed to detect small molecules, nucleic acids and proteins. Such advances may constitute the most promising approach for electrochemical biosensor development by exploiting techniques that depend on binding-induced changes in receptor physics. In this context, nucleic acids are furthermore amenable to integration with DNA/RNA nanostructures and thus stand to benefit from significant advances made by the rapidly converging fields of nucleic acid nanotechnology and aptamer research.

Overall, two distinct techniques exist for biosensor development: receptor-based sensors that depend on adsorption-linked physical changes, and alternatively, techniques depending on binding-induced changes in receptor physics. Notably, electrochemical sensors exploiting adsorption-based signal induction typically fail when challenged in biofluids, requiring the need for sample processing or enrichment methods that add to device complexity and cost. In contrast to adsorption type sensing (measured by changes in mass, charge, or optical refraction), nature inspired binding-induced events can be exploited instead (changes in the receptor's confirmation, oligomerization states, or dynamics). Biosensors based on binding-induced conformation changes are relatively recent and have proven advantages in terms of specificity in complex matrices. Most crucially, techniques relying on binding-induced conformational changes, or ‘structure-switching’ better meet the criteria to support feedback control for advanced real-time biomarker monitoring which is expected to advance electrochemical diagnostics toward theranostic applications and pave the way toward increasingly sophisticated POC technologies. In the EAB structure-switching approach, an electroactive redox molecule is typically conjugated to an aptamer terminus at either 3 or 5′ ends, which is then either elevated away from- or brought towards the electrode surface (to which the aptamer is immobilized) upon target binding. The change in distance between the redox probe and surface alter electron transfer efficiency which is read as a change in current passed through the electrode, most commonly by voltametric methods such as square-wave-voltammetry. This approach is recently adopted for detection of SARS-CoV-2 spike (S) protein displayed on a virion surface with accurate readouts generated in as little as 5 minutes. Idili and co-workers likewise have developed a biosensor for the same target using aptamers designed to undergo binding-induced conformational changes but using well established gold-thiol chemistry to immobilize aptamers onto a working electrode. CRP-targeting aptamers have been used to construct various sensing platforms.

However, no such structure-switching approach for EAB based CRP detection has been developed yet.

Therefore, the present invention provides an electrochemical sensing platform based on cheap, commercially available screen-printed gold electrodes, functionalized with a methylene blue modified CRP-targeting aptamer. Upon complexing with CRP, the aptamer is induced to undergo a conformation switch leading to dose-dependent increase in measured current signal in the microamp range. It is demonstrated that the operation of this method in both BSA doped PBS buffer, 50% diluted human serum and undiluted human blood with no loss in sensitivity or selectivity. Furthermore, the nanomolar sensitivity can be achieved by incubation with CRP in as little as 10 minutes.

The invention describes a biosensing method using aptamer for electrochemical detection of C-Reactive protein (CRP). Aptamers featuring an electroactive redox molecule are immobilized onto the surface of a 3.3×1 cm screen printed (gold) electrode. Upon target recognition and binding, the distance between the aptamer-tethered electroactive molecule is reduced to produce a significant, quantifiable and specific signal-ON event which can be interrogated easily using a potentiometer and laptop or any suitable computer/tablet or mobile phone set up with the appropriate software. The biosensing platform exceeds the clinically relevant cut-off for CRP detection by 10-20×, 1-10 minutes in 50% diluted human serum or undiluted human blood.

Diagnosis and timely treatment for pathologies where CRP is used as a biomarker, such as sepsis, is currently limited in the clinic due to lengthy times required to conduct assays. The presented platform provides significant advantages by providing highly sensitive readouts in significantly faster times, without the need for complex sample processing and/or enriching. Furthermore, the method avoids the requirement for multiple reagents and/or labels that are typically required to conduct assays, e.g., ELISA. The platform can also be operated by the non-specialist, which is expected to be a major focus point for translation into an optimized point-of-care device. POC diagnostic technology is expected to significantly improve clinical decision making by abrogating the need for centralized laboratory-based tests that often suffer delays and back-logs. Additionally, as CRP fluctuation is indicated in numerous pathologies, not all of which are life-threatening; therefore, in some embodiments, the biosensing method may have prospects for broad application in development of hand-held sensor, for use by the general public.

Furthermore, the method completely abrogates the need for labels, reagents and centralized lab-based tests by exploiting miniaturized electronic sensors to produce readouts in a few minutes. The method is amenable to operation by the non-specialist and can be integrated to work on mobile phones, laptops or using any suitable computer interface to display results, making the miniaturized sensors for CRP detection potential for use by the public for the reasons outlined above. As the method exploits the use of DNA-based receptors, a significant cost reduction is also expected in comparison to well established antibody-based technologies such as ELISA. Compared to antibodies, DNA has improved overall chemical and thermal stability profiles, improved shelf-life and can match sensitivity in terms of binding affinities where such components are used as receptors in biosensing devices.

In summary, the present method supersedes the adsorption-based principles traditionally used to construct antibody-based sensors by exploiting instead, binding-induced changes to receptor (aptamer) confirmation to induce signal generation. In this way, the sensing method of the present invention is more amenable to operation in complex biofluids (blood or serum) without dilution and so can avoid the need for additional reagents or sample processing to reduce device complexity, times required to produce results and thus associated costs. The sensing method is also more amenable to real-time monitoring as it avoids false positives commonly associated to adsorption-based biosensing methods that typically fail under such conditions (requiring the need for time-consuming sample processing by enrichment or dilutions that are difficult to standardize).

### EXAMPLES

### Materials and Methods

### Aptamer Immobilization on BT-250 SPEs.

Phosphate buffered saline (PBS) 10× is purchased from Thermofischer. Tris(2-carboxyethyl)phosphone-HCL (TCEP-HCL) is purchased from ThermoFischer. 6-Mercarpto-1-hexanol (6-MCH) as dry powder, 99% is purchased from Sigma-Aldrich (Merck brand). Endotoxin-free ultrapure water is purchased from Sigma-Aldrich. Sulfuric acid (H₂SO₄) ACS reagent, 95.0-98.0% is purchased from Sigma-Aldrich (Merck). Potassium ferrocyanide trihydrate K₄[Fe(CN)₆]·3H₂O and potassium ferricyanide K3Fe(CN)6]>99.95% are purchased from Sigma-Aldrich (Merck). Bovine serum albumin lyophilized powder>96% is purchased from Sigma-Aldrich (Merck). Serum from Human male AB plasma (USA origin, sterile-filtered) is purchased from Sigma-Aldrich. C-reactive protein ‘CRP’ (ab167710, HEK 293 expressed >95% purity, <1.000 Eu/μg) is purchased from abeam plc. CRP targeting aptamer, with the following sequence: GGCAGGAAGACAAACACGATGGGGGGGTATGATTTGATGTGGTTGTTGCATGAT CGTGGTCTGTGGTGCTGT (SEQ No. 1), is purchased from IDT, Singapore with 5′ Thiol modifier (C6-S—S) and 3′ methylene-blue modifier via C7 spacer. Control sequences used for this study are likewise purchased with the same modifications from IDT including an IL-6 targeting aptamer: GGTGGCAGGAGGACTATTTATTTGCTTTTCT (SEQ No. 2) and the CRP targeting aptamer with primer flanking regions removed: CGATGGGGGGGTATGATTTGATGTGGTTGTTGCATGATCG (SEQ No. 3). Screen-printed BT-250 electrodes with gold working electrode, platinum and silver counter and reference electrodes respectively are purchased from Metrohm Dropsense via Labware HK.

First, SPEs are rinsed thoroughly with milli-Q purified water for at least 10 seconds. Then, electrodes are treated with 0.5M H₂SO₄ and subjected to electrochemical cleaning via cyclic voltammetry. Briefly, CV sweeps are conducted with 0.1V/s applied scan rate across a voltage range of 0.01-1.25V for 10-20 scans. CRP-targeting aptamer (10 μM) is incubated with TCEP at 200× molar equivalents in ultrapure H₂O for one hour to reduce disulfide bonds. Following reduction, the aptamer is diluted to a final concentration of 2 μM into 2×PBSM buffer (2 mM magnesium) and placed onto dried electrodes in a humidity chamber and incubated overnight at room temperature. The following day, reduced aptamer containing solution is rinsed off using PBS(1×) followed by back-filling with 6-MCH (6 mM) diluted in PBS(1×) for 2-3 hours at room temperature. For recovery assays and general storage, sensors are washed thoroughly with PBS(1×) and stored in a humidity chamber at 4 degrees.

### Determination of the Aptamer Surface Density

UV spectroscopy is used to estimate the surface density of chemisorbed aptamers. The absorbance of methylene blue modified aptamer containing solution is measured by nanodrop at 260 nm, before and after immobilization. The absorbance change is used to calculate the estimated number of aptamer probes per mm² gold working electrode surface. The measurements are replicated from three separate electrodes with the reported value constituting mean value with standard error. Electrochemical impedance spectroscopy ‘EIS’ is used to qualitatively gauge density of aptamer and aptamer: MCH-SAMs as inferred by size of the semicircles in generated Nyquist plots, which relate to charge transfer resistance at the electrode surface. EIS is conducted in 5 mM potassium ferri/ferrocyanide solution prepared in PBS(1×) buffer by sweeping from 0.1 Hz to 1 kHz against an ‘open-circuit potential’. Applied alternating potential amplitude E_{ac} is set to 10 mV with direct potential set to 0V.

### Detection of CRP

A solution of CRP at different concentrations is prepared in either PBS doped with BSA to a final concentration of 1 mM or human serum diluted with PBSM (1×) 2 mM magnesium. Samples are incubated for 5-10 minutes prior to measurements made with a commercially available potentiostat. Applied voltametric parameters used for measurements are as follows; starting potential of −0.6V, ending potential of 0.0V, E steps of 1 mV, applied amplitude of 50 mV and applied Frequency of 1 KHz. Applied frequencies are adjusted from 0.1 to 1 KHz to investigate signal response gain in separate experiments. For real-time assays, measurements are made at 1 KHz in human serum (50%) with 10 second intervals.

$$y = {V}_{mαx} ⋆ {x}^{n} / ( {k}^{n} + {x}^{n} )$$

### Specificity Assays and Recovery Assays

Sensor specificity is tested by conducting dose response assays in either 1 mM BSA doped PBS(1×) buffer, or 50% human serum diluted with PBSM(1×). Specificity is additionally investigated by spiking in designated concentrations of plasmodium lactate dehydrogenase ‘pfLDH’ and procalcitonin ‘PCT’. Both proteins are expressed in BL21 E. Coli with a 6× C-terminal His tag (data not shown). Sensor ‘recovery’ is described as the sensor performance assessed after initial use and is calculated from n=4 separately prepared sensors.

### Data Acquisition and Analysis

The recorded current signals obtained from electrochemical experiments are extracted from PSTrace software (supplied with the commercially available Palmsense4 potentiostat) into Microsoft excel and Origin for linear regression analysis and fitting to the Hill-Langmuir (dose response) isotherm, the equation for which is shown below (Eq 7) where Y is the output response, X is in the input concentration, Vmax is the maximum velocity of the reaction, n is the hill coefficient and K is the binding constant. The Levenberg-Marquardt algorithm is applied to solve non-linear fitting for the Hill plot. Limits of detection are calculated using the usual formula of 3.3×standard error of the slope over the slope value. Relative response is calculated as a percentage value by taking the difference between the current produced after and before incubation with the target at respective time points, i.e., ([Response current at t=x]−[Baseline current at t=x])/[Baseline current at t=x]), where t=x denotes the incubation duration. All Electrochemical measurements are recorded using a Palmsense4 potentiostat with an MUX8 multiplexer and the supplied software PSTrace version 5.9 (Houten, Netherlands, supplied by regional distributor Redmatrix, China).

### Example 1. The Development of an Aptamer-Based Biosensing System for Rapid, Single-Step Electrochemical Detection of CRP

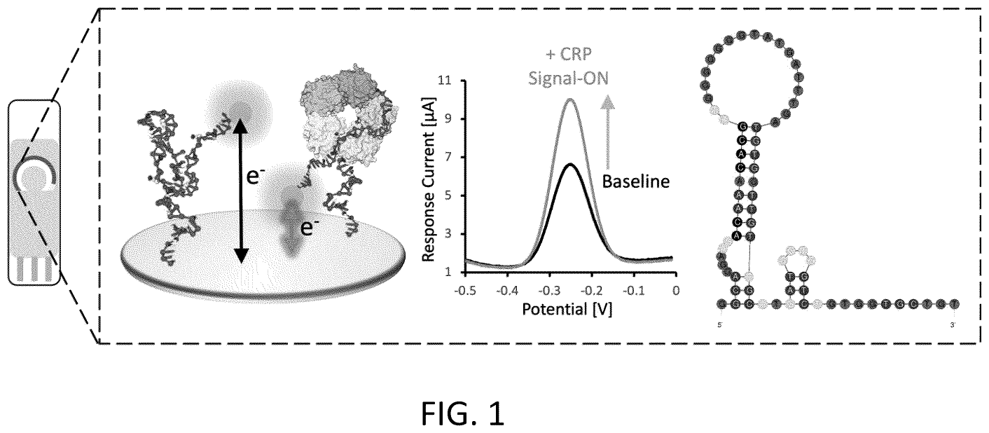

The sensing platform is developed using cheap, commercially available screen-printed gold electrodes functionalized with a redox-tagged CRP-targeting aptamer sequence (FIG. 1). Here, the aptamer DNA sequence is modified with a 5′ disulfide group via a six-carbon spacer.

Well established gold-thiol chemistry is used to immobilize the aptamer onto the electrode surface by reducing a terminal disulfide group in a single-step chemical reduction (FIG. 1) followed by “back-filling” of the electrode surface with alkane-thiols to prevent non-specific adsorption of non-target proteins and/or interfering chemicals commonly found in biofluids. Based on assessment of such studies, a concentration of 2 μM DNA is selected to generate a binary self-assembled monolayer ‘SAM’ via backfilling with 6MCH. Prior to immobilization, gold SPEs are subjected to electrochemical cleaning via cyclic voltammetry, which revealed a substantial increase in the gold reduction and oxidation peak heights indicative of an increased electroactive area.

Routinely used electrochemical interrogation methods including impedance spectroscopy are used to verify successful fabrication of the sensor (FIG. 1). Potentiometric electrochemical interrogation also verifies successful fabrication of the sensor as indicated by the observation of an obvious current peak in the range expected for and induced by electron transfer provided by the aptamer terminal conjugated redox probe, methylene blue (FIG. 1). Upon binding to CRP, the aptamer is induced to undergo a conformation switch leading to dose-dependent increase in measured current signal as assessed by interrogation via the potentiometric method Square Wave Voltammetry (SQWV). It is evidenced that the applicability of this method for nanomolar detection of CRP in idealized buffers (PBS buffer) and diluted human serum with no loss in sensitivity or selectivity.

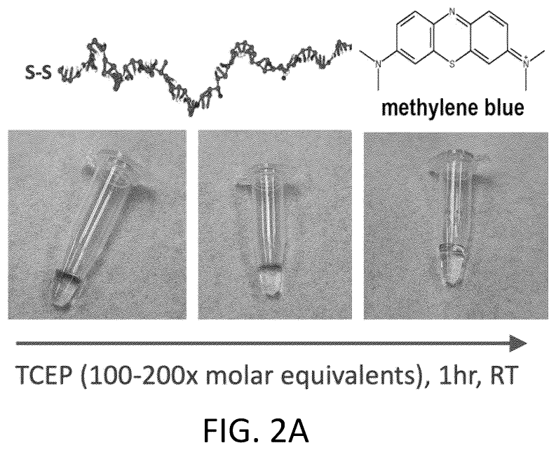

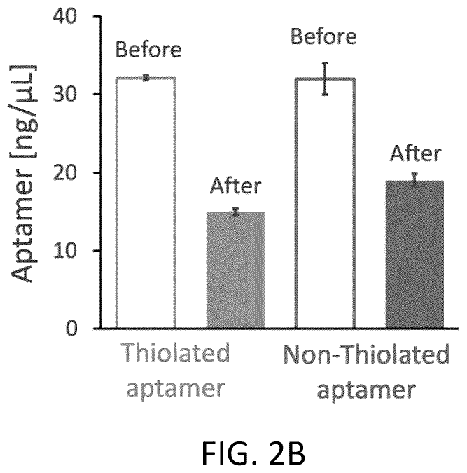

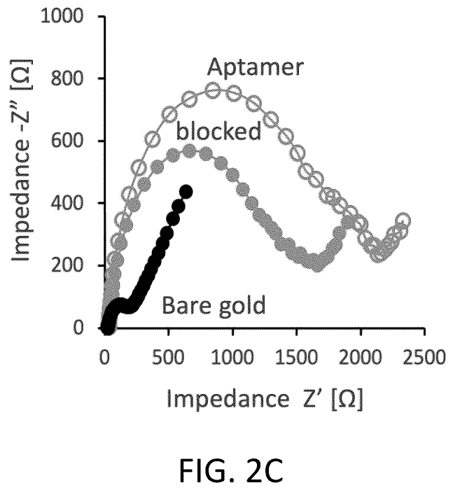

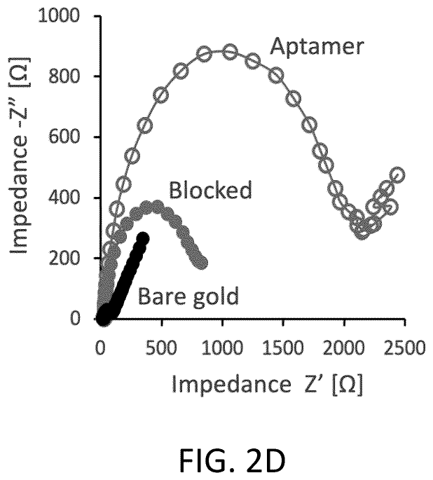

The use of MCH to generate mixed SAMs (with nucleic acids) is well established and is detailed elsewhere. Numerous studies have investigated the relationships between aptamer concentration used for immobilization and packing density of the resulting SAM with findings generally showing that 0.5-2 μM is optimal. Notably, a recent investigation shows that the most significant contributing factor for EAB optimization is produced not by optimizing aptamer concentration used to generate the SAM, but depended primarily on applied electronic parameters used to interrogate faradaic electrochemical processes. The qualitative experiments show no increase in the observed current peak height produced by methylene blue above 1-2 μM DNA. Baseline values across a range of aptamer concentrations from 0.1 to 5 uM are recorded over time and showed little drift in PBS buffer. Analysis of the aptamer solution by UV 260 nm absorbance before and after immobilization onto gold SPEs reveals a concentration loss of 53.3% which is used to estimate aptamer packing density on the electrode surface (FIG. 2B). It is estimated that a packing density of 4.02*10¹⁴ aptamer molecules per mm². It is worth noting however that aptamers may also immobilize onto the platinum counter electrode surface, albeit with reduced efficiency. Furthermore, this value does not consider the portion of displaced aptamers following MCH backfilling, which is revealed to be significant as inferred by Electrochemical Impedance Spectroscopy (EIS).

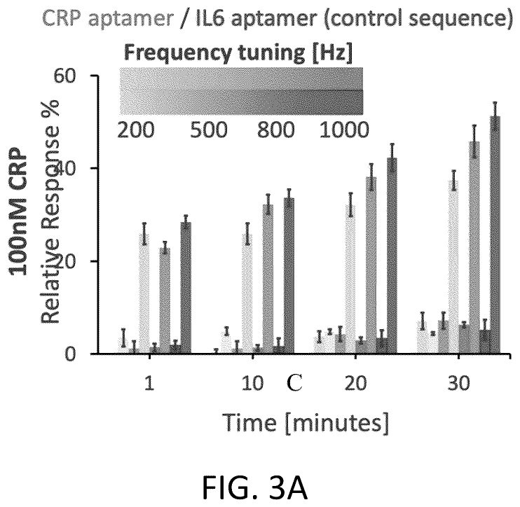

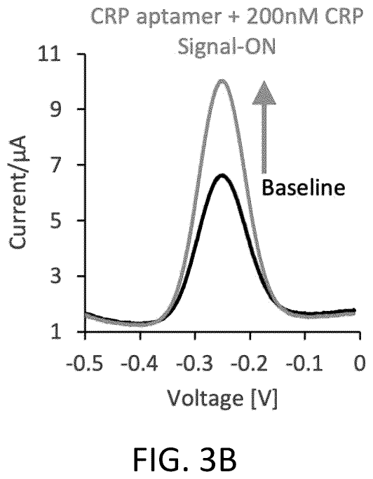

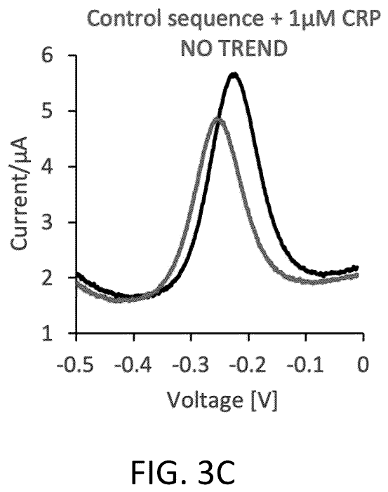

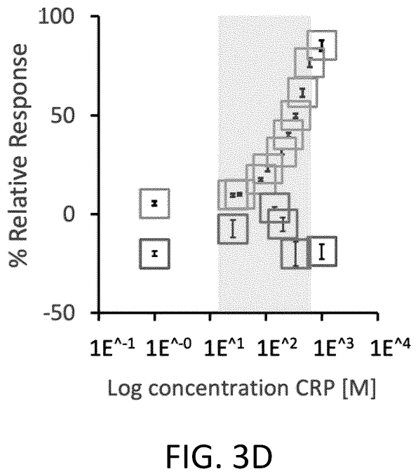

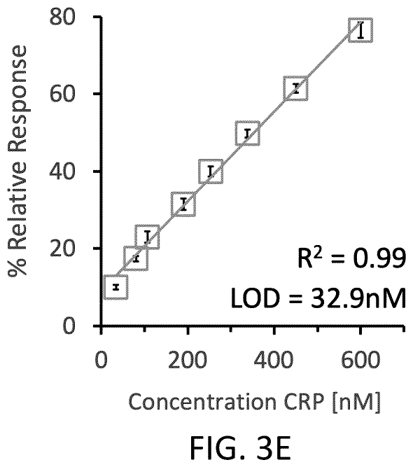

Nyquist impedance plots are generated to monitor formation of the ap-tamer/MCH-SAM by visual interpretation of the semicircular curve size and area which directly relate to charge transfer resistance (Rct) across the electrode-solution interface (FIGS. 2C-2D). Notably, it is observed a small yet significant decrease in Nyquist plot semicircle size following back-filling with MCH. By comparison, a control sequence lacking terminal disulfide modification showed a significantly greater decrease in Rct (as inferred from the Nyquist semicircle size), suggesting that thiolated aptamers are more robustly immobilized in comparison to their non-thiolated analogue. Finally, aptamer immobilization is conclusively verified by observation of an obvious current peak across the associated voltage range for methylene blue (FIGS. 3A and 3B).

### Example 2. Specificity and Sensitivity of CRP Aptasensor in BSA Doped PBS Buffer

Refer to FIGS. 3A-3E for the following descriptions. To assess specificity of the CRP-targeting aptamer, initial assays are conducted in PBS (1×) buffer supplemented with bovine serum albumin ‘BSA’. Albumin is fixed at a concentration of 1 mM to reflect slightly higher concentrations than those found in serum. Control sensors are prepared in the same fashion as with test sensors using an excised version of the CRP-targeting aptamer sequence without flanking primer regions (FIG. 3D). Dose-response plot plotting confirmed feasibility of this CRP-targeting aptamer for structure-switching induced signal induction as assessed via SQWV and revealed a linear response range spanning one order of magnitude, from approximately 10-500 nM CRP. A Limit of detection ‘LOD’ of 105.4 pM is calculated using linear regression, using n=3 independently prepared sensors. Notably, the lack of response induced by the control sequence confirmed the aptamer selectivity in this buffer system. A response plateau is not observed at the maximum concentration tested (500 nM CRP) which does not exceed, as this concentration exceeds the clinically relevant window for CRP in serum. Here, CRP concentration is reported in molar units and calculate values based on the pentameric isoform weight of 115-120 kDa, as is reported by the commercial supplier and elsewhere.

### Example 3. Sensitivity and Reusability of CRP Aptasensor in BSA Doped PBS Buffer

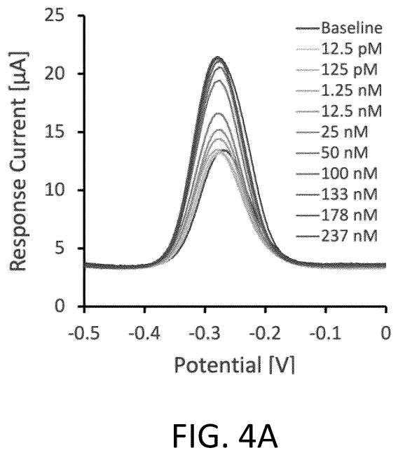

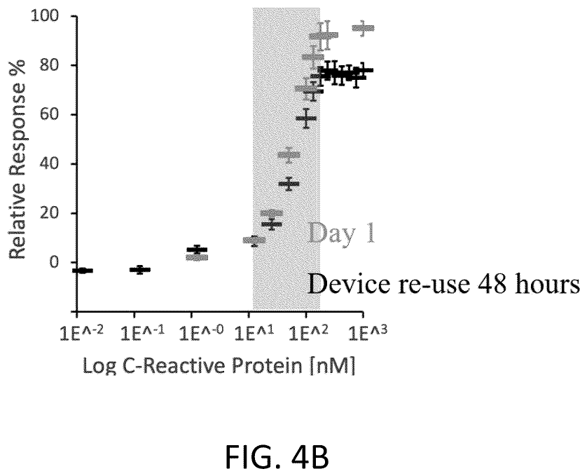

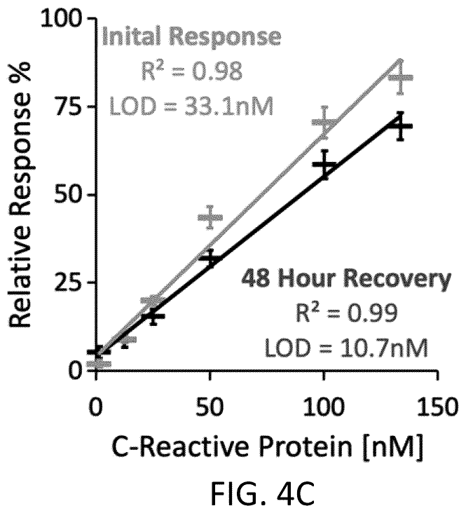

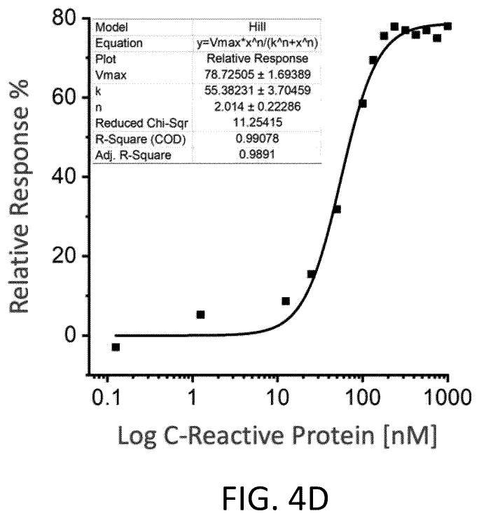

Refer to FIGS. 4A-4D for the following descriptions. To assess reusability of the prepared aptasensors, dose-response assays are conducted in BSA doped PBS buffer 48 hours after initial testing. Magnesium is used to supplement PBS buffer to more closely match the ion content found in serum with observations showing little difference between results obtained from the initial screening assay. Reuse of n=3 independently prepared sensors after 48 hours reveals a partial loss in sensitivity, the cause of which might be explained by numerous factors including desorption of aptamers from the electrode surface, changes in ambient temperature or inaccuracies associated with sample preparation through human error. Despite these uncertainties, linear range observed for used sensors is broadly maintained. For used sensors, the LOD calculated from linear regression is shown to be approximately 363 pM which matched well with an LOD value of approx. 374 pM derived from non-linear regression fits to the Hill-Langmuir (dose response) function described in section 2.6. Non-Linear fitting is achieved with good fitting (adjusted R₂=0.99). A dissociation constant (K_{d}) of 55.38±3.7 nM is estimated from the non-linear 48-hour recovery data which is well within the clinically relevant cut-off range for CRP detection (50 mg/L). Here, concentration is provided in molar units, calculated using the CRP pentameric isoform weight of 120 kDa.

### Example 4. Sensor Performance and Optimization

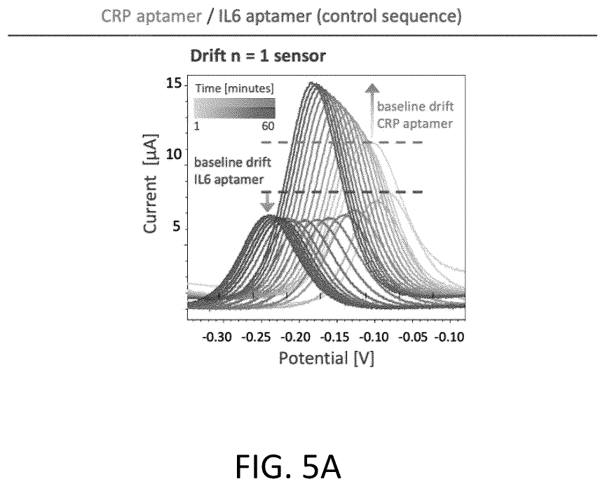

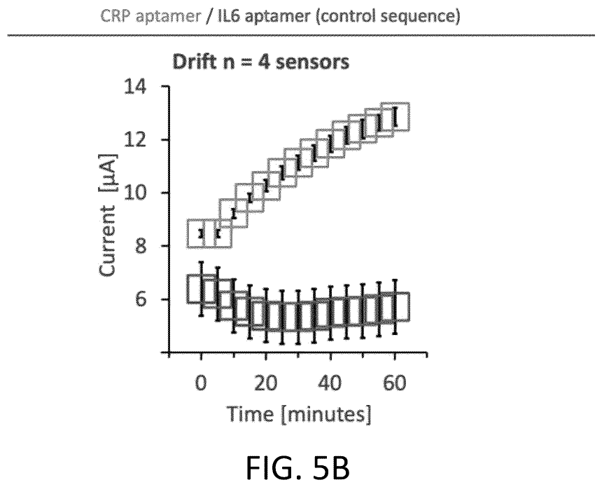

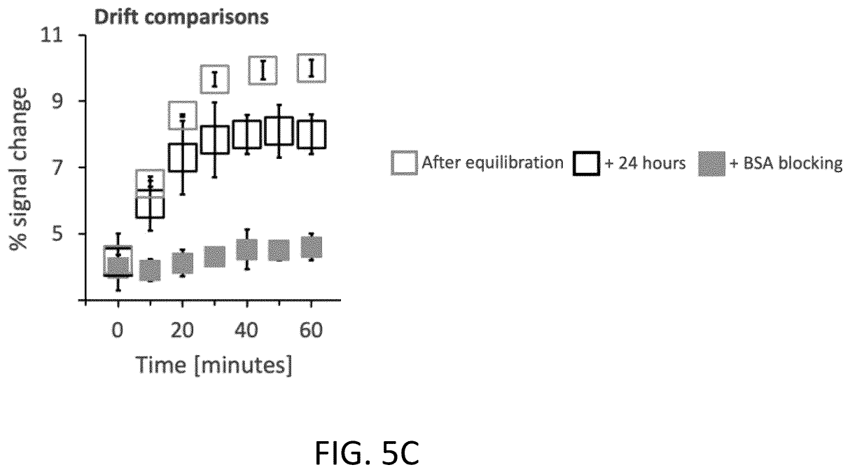

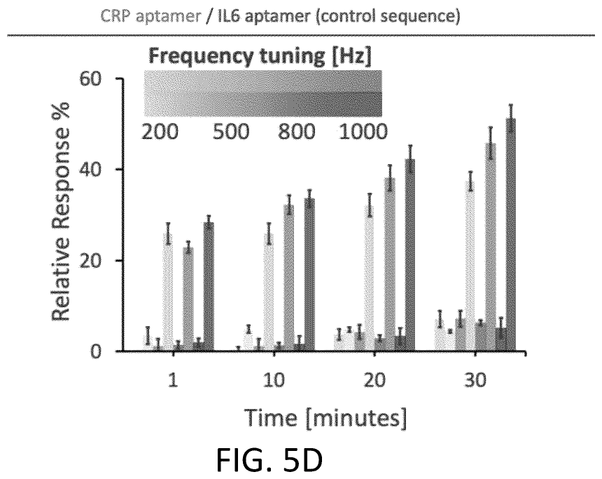

Sensor stability was assessed by taking measurements of the baseline response over one hour in PBS diluted human serum. Surprisingly, a significant drift is observed for the CRP-targeting aptamer but not the interleukin-6 control sequence (FIG. 5A). For the CRP aptamer, the baseline response is observed to increase in a linear fashion for at least 30 minutes (FIGS. 5A-5B). In comparison, dummy sensors constructed with the control IL6 targeting aptamer show a relatively stable baseline which is maintained after testing cycles (INSERT SI). Notably, larger overall baseline response values are observed for the CRP aptamer, which might be explained by the increased length of this sequence relative to the control IL6 aptamer (FIG. 5B). In comparison to the control sequence, the longer sequence CRP-targeting aptamer is more conformationally free to fold onto itself and bring the terminal conjugated redox probe closer to the electrode surface, hence inducing faster electron transfer rates which correlate with a larger signal induction in the form of measured current output. The shorter control IL6 aptamer is likely less able to undergo such folding and may therefore be expected to remain relatively flexible and ‘upright’ hence increasing distance between the probe and electrode surface to produce smaller current induction. Crucially, baseline drift is shown to be largely abrogated by incubating the sensors in BSA containing buffer (1 mM in PBS 1×) for 30 minutes prior to kinetics-response measurements (FIG. 5C).

Response induced by incubation with 100 nM CRP is later shown to increase over time, attributed to this drift effect (FIG. 5D), hence confirming the importance of this secondary blocking step after initial ‘backfilling’ with alkanethiols. Indeed, various studies have reported the importance of sensor stabilization and blocking with proteins after mixed monolayer SAM formation. X et al., argue that additional blocking steps with proteins in this context may be required to fill in larger ‘voids’ left on the sensor surface after SAM formation, which may be caused by aggregation (of oligonucleotides or alkanethiols) induced by electrode surface defects. Notably, SPEs have much rougher surface topology in comparison to thin-film electrodes, which may explain the need for sufficient blocking steps to stabilize sensors built on such materials as demonstrated here. Finally, by tuning the applied frequency used for SQWV measurements, it is shown that significant enhancement to signal gain is possible (FIG. 5D). The selectivity of the CRP targeting aptamer employed here is furthermore confirmed by lack of response induced by the control sequence.

### Example 5. Sensitivity and Specificity of CRP Detection in 50% Diluted Human Serum

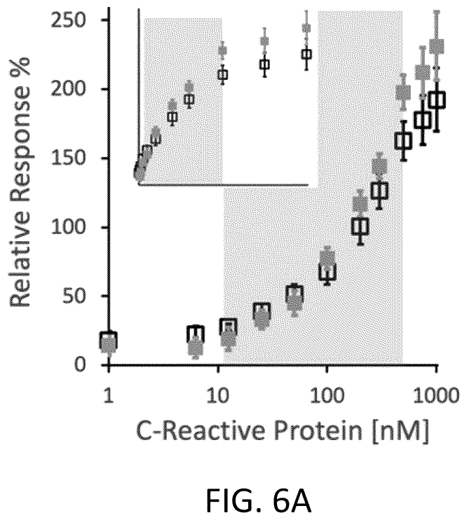

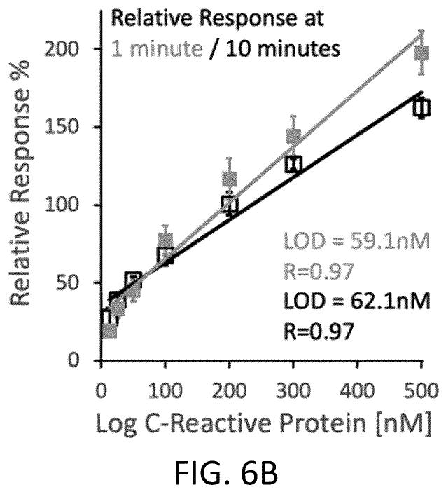

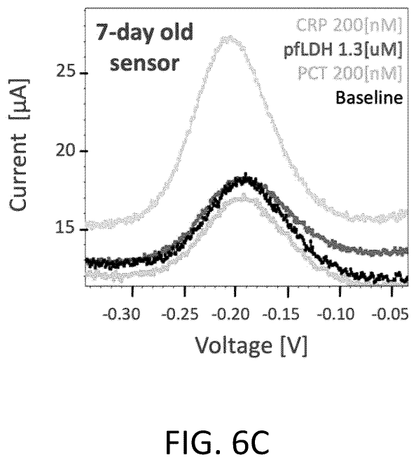

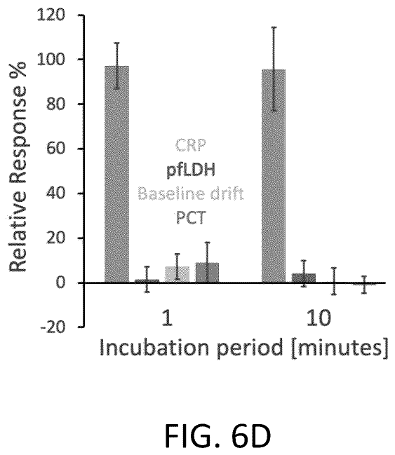

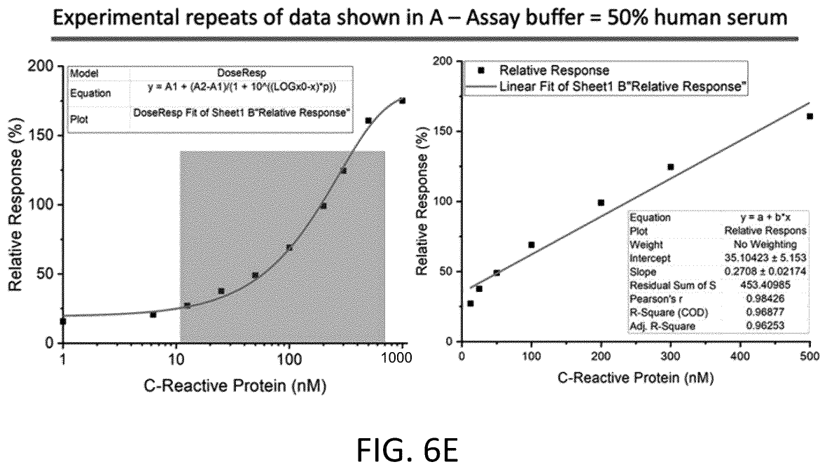

Refer to FIGS. 6A-6D for the following descriptions. To test the applicability of this sensor in clinically relevant conditions, dose-response assays are conducted initially in 20% diluted human serum. Noticeably, the linear range observed is broadly maintained with no significant difference observed for the calculated LOD (FIGS. 6A-6B). A value of 105.5 pM was obtained for the sensor using sample incubation periods of between 5-10 minutes. The sensitivity reported here thus exceeds the clinically relevant cut-off for CRP by 3-orders of magnitude, or by approximately 4000× and equates to a weight/volume concentration of approximately 12.6 μg/L. Notably, the apparently well-maintained sensitivity achieved here, upon changing from simplified assay buffers to complex biological fluids, is observed across numerous studies where aptasensors are constructed using the structure-switching approach. This may be attributed to the signal induction dependence on binding-induced changes to receptor physics, as opposed to sensors (including aptasensors) that depend on adsorption-based signal induction mechanisms. The latter are notoriously prone to false-positives and response dampening, in addition to baseline drift commonly attributed to non-specific interaction with abundant proteins and interfering chemicals found in complex sample matrices. Here, the structure-switching capability of a DNA aptamer is exploited to induce a signal-ON event which is furthermore advantageous over the Signal-OFF (suppression) type response mechanisms typically exploited for adsorption-based sensors.

Furthermore, it is shown that nanomolar sensitivity can be achieved in spiked human serum samples in as little as 5-10 minutes. Limits of detection ‘LOD(s)’ for the sensor (20-60 nanomolar) are established using triplicate experimental controls via linear and non-linear regression analysis from dose-response assays. The device can be re-used at least once with remarkable sensitivity sustained within the nanomolar range up to 48-hours after initial use (FIG. 2B—bottom panel). The LOD for this sensor exceeds the gold-standard clinical cut-off for CRP detection (50 mg/L) by a single order of magnitude, or by approximately 10×, equating to an LOD of approximately 5.4 mg/L.

### Example 6. Realtime Sensing Capabilities Mimicked by Droplet Injection of CRP

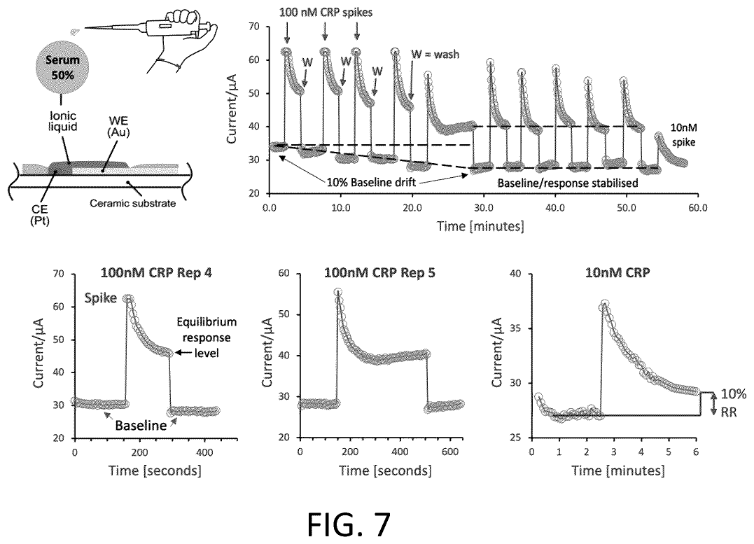

In combination with clinically relevant sensitivity, rapid detection times displayed by the sensor (5-10 minutes) offer strong prospects for translation into a point-of-care technology. CRP is a commonly used biomarker for diagnosis and monitoring of a wide range of pathologies in clinical settings. Work-around times however still typically exceed 1-2 hours in the clinic, constituting serious limitations regarding acute-phase pathologies such as sepsis, where CRP can be used as a sensitive indicator. Large cohort studies have found as much as a 10% increase in morbidity prognosis per hour for patients with sepsis between admission and administration of antibiotics. The biosensing approach is based on the relatively new method of exploiting binding-induced changes to receptor (aptamer) physics which is known to be significantly less prone to false-positives commonly associated with traditional and better-established methods exploiting adsorption-linked physical changes to receptors. Crucially, the improved selectivity of the present approach posits this platform as highly suitable for real-time biosensing, which promises additional and significant commercial value by providing a means to expand device development toward theranostic applications where drug administration can be adjusted and controlled via continuous response-adjustments. Therefore, the tentative real-time assays are conducted. Briefly, the recombinant (human cell expressed) CRP is spiked into droplets of human serum on screen printed electrodes. Rapid response is observed to spike and plateau after approximately 60 seconds. The process is repeated with washing of the electrode surface over 10 cycles with 100 nM CRP, followed by a final 10 nM spike that is easily distinguished. The results show that rapid response is possible within the sensor dynamic range (FIG. 7). Stable response signals are observed after an initial baseline drift period over 30 minutes, the exact cause for which is under investigation. 10 nM CRP is easily detected and distinguished after 10 cycles of 100 nM CRP detection in human serum, suggesting good sensitivity recovery. In context, 10 nM CRP using a 50% human serum dilution constitutes an approximate 40× sensitivity over the clinically relevant cut-off of approx. 415 nM CRP as calculated using the protein pentameric molecular weight of 120 kDa.

### Example 7. Device Re-Usability and Additional Selectivity Verification

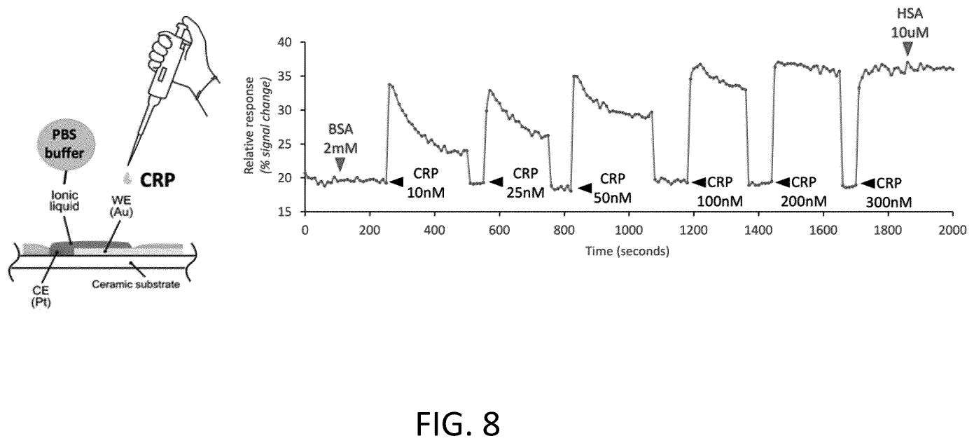

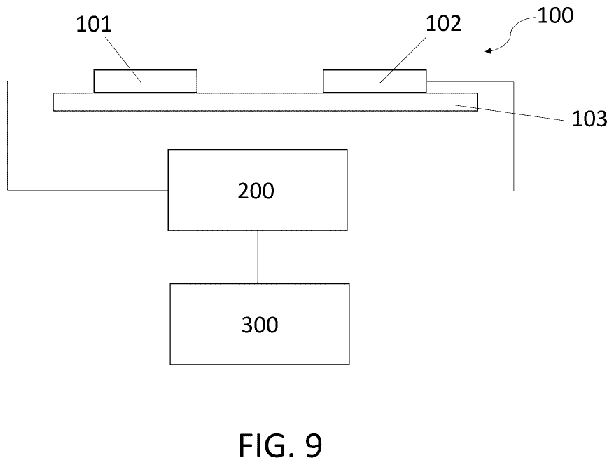

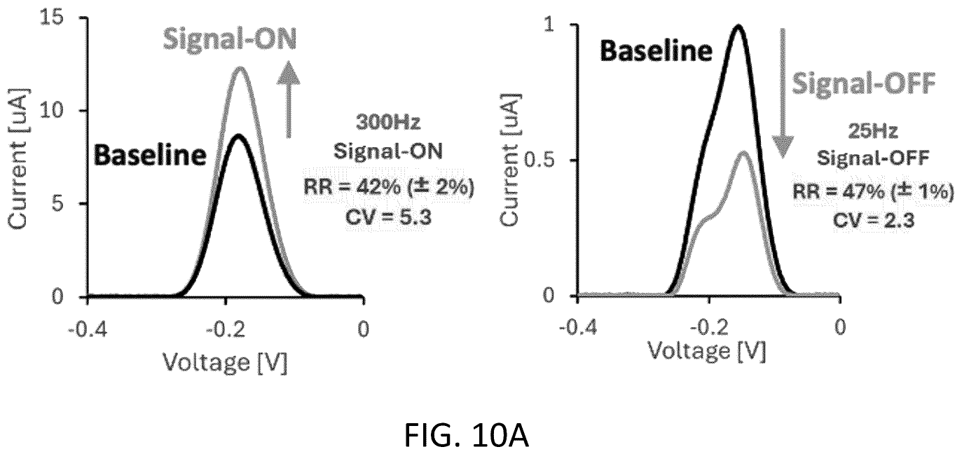

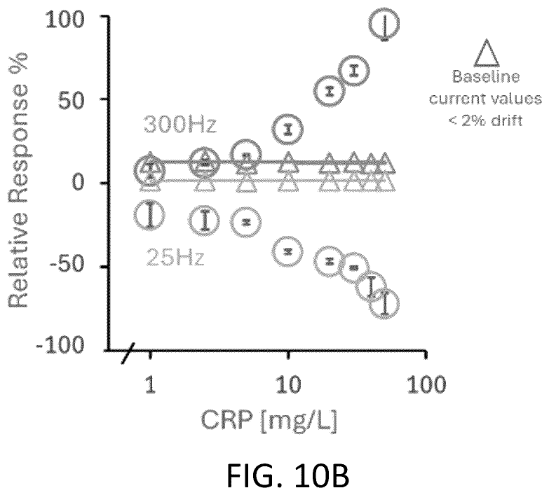

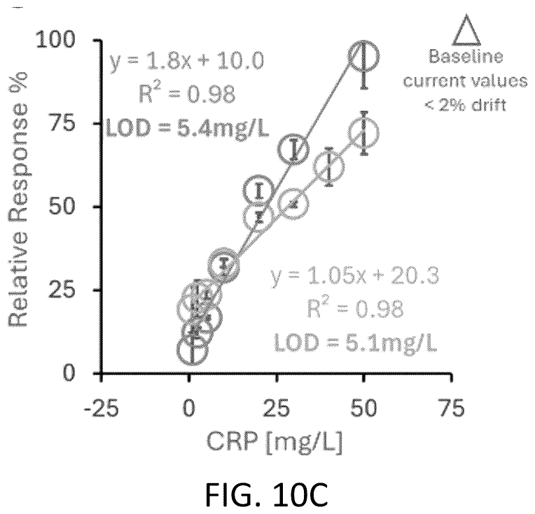

As shown in FIG. 8, the injection of controlled CRP doses into an electrode-surface confined droplet of assay buffer (PBS) confirms a dose-response increase in signal output generated by square wave voltammetry interrogation at 10 second intervals. The initial response spike is caused by injection of the analyte. Subsequent stabilisation of the response signal via this testing method occurs over 2-3 minutes. Crucially, significant concentrations of bovine serum albumin, and human serum albumin produce no observable deviation in response, confirming excellent selectivity against serum endogenous proteins that are well known to produce false positives in other sensors formats.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A single-step, reagentless and reusable electrochemical biosensor system for detecting C-reactive protein (CRP) in a specimen, comprising:
a working electrode with redox-tagged CRP-targeting aptamer immobilized on the surface of the working electrode and;
a counter-electrode back-filled with a backfilling reagent to prevent non-specific adsorption;
a flat substrate surface for mounting the electrodes and the specimen;
a current signal recorder connected to the working electrode and the counter-electrode; and
a data processor for receiving the current signals from the current signal recorder and processing the current signals;
wherein the CRP detection sensitivity of the electrochemical biosensor system is in the range of 1 to 500 nanomolar.

2. The system of claim 1, wherein the working electrode is a gold electrode.

3. The system of claim 1, wherein the counter-electrode is a platinum electrode.

4. The system of claim 1, wherein the redox tag of the CRP-targeting aptamer is methylene blue.

5. The system of claim 1, wherein the backfilling reagent comprises alkane-thiols.

6. The system of claim 1, wherein the substrate is ceramic.

7. The system of claim 1, wherein the redox-tagged CRP-targeting aptamer is immobilized on the surface of the working electrode by gold-thiol interaction.

8. The system of claim 1, wherein the detection time for the CRP is no longer than 10 minutes.

9. The system of claim 1, wherein the system is reusable for more than 1 time for dose-response assays.

10. A single-step and reagentless electrochemical biosensing method for detecting CRP in a specimen using the electrochemical biosensor system of claim 1, comprising:
collecting a specimen from a subject;
obtaining a biological fluid from the specimen;
subjecting the biological fluid obtained from the specimen to the electrochemical biosensor system; and
determining the presence or absence of CRP in the specimen by observing the current signals received by the data processor.

11. The method of claim 10, wherein the biological fluid comprises serum, plasma, cerebrospinal fluid, interstitial fluid, saliva, and human blood.

12. The method of claim 10, wherein the fluid is CRP-positive if the current signal received increases or decreases in a dose-dependent manner.

13. The method of claim 10, wherein the detection time for the CRP is no longer than 10 minutes.


SEQUENCE LISTING (3 sequences)

SEQ ID NO: 1 - DNA, 72 bp, synthetic construct
  source 1..72: mol_type = other DNA
ggcaggaagacaaacacgatgggggggtatgatttgatgtggttgttgcatgatcgtggtctgtggtgctgt

SEQ ID NO: 2 - DNA, 31 bp, synthetic construct
  source 1..31: mol_type = other DNA
ggtggcaggaggactatttatttgcttttct

SEQ ID NO: 3 - DNA, 40 bp, synthetic construct
  source 1..40: mol_type = other DNA
cgatgggggggtatgatttgatgtggttgttgcatgatcg